SEQUENCE LISTING (11 sequences)

SEQ ID NO: 1 - DNA, 1550 bp, Oryza sativa
  CDS (77)..(1465): DNA sequence encoding OsSRO1c
catttctccacctgcacatctgcgcgcgcgcgcgggagagcagaggcggcgagagagatccgggtcgggaggggtgatggacttctccggcgacgtcaagccggcgatccaccggccctctgtcgcggcggcacgcgggggagggaacggtggggcgatcccgctcctgcgtgggtggcaggcgttccggaggagcggcgcgccggcgaggctcctctgcttcgagggcggcgcgtgggcggacgtcgcgggcgaggtggtggggctgctgcggcgggcgttcatggaggggaaggccgtttgcgaggccgcctgcggtgggagggtgttcctgttcgacttcatgcggatggttcggatcgatgaggccaccgccgaggaggccgcgctggggtggatcgacgaccgcggcgcgtgcttcttcccggctcccgagggcgggaggaagaggaagagggagagggacgcggggtcggaggtgaagggggaggatcggcggcggcggcagccggcggcggaggaggaggacggggacgaggcgtcgtccggcgtggaggagcggtccggggagagccgccccgaggcggatgagcccgacaggaagaaggcgcgcgggacgttgtgggggaaggcggtgaggctggacgaggcggacaagttctacaaggtggtggagaagctcttcgtcagccggatggctcccgtggcggcggcccgcggcgtggcgatcacggcggtgcacaaggtcgcgcaggggccccgggcaagagccttccatctgcagggacagctccttgccgctgctcgcggcgtcggcgatggcagcaacgccaagttcgcgtggtacggcgcgccggcggcggatgtggccgcggcggtggagcacggcttcgggaggacgaacgggcagtttctcggcgggcgcgcacacggcgacggcgttcacctttcgccgccgcagtaccctcacgctagtgcgatgctgaccaagccagacgagaatggcgaggcacacatcgtgctgtgccgcgtcctgatgggccgtccagaggccgtccctgccagctcaccccaattccaccccagcagcgacgaatacgacagcgccgtcgacaaccttgagaatccgcggtggtacgttgtatggagcacagacatgaacaccaggatcctcccagagtacgtggtcagcttcaggtggcccaacctgccgcagatggaaggatcatcggggttgggatcgaagctgaagaagccatcaccagcagctactcgcgacatgttccctatgcttctgacggagatccagcggttcgttccatccccgaagctgcagactttgcagaggacgtacaactgcttcaagagaggacagatgaagaaggaccagttcatccggttcttgcgctcccacatcggcgacaatgtgttgaccaccgtggccaagaaactccgagggtactagtgcaaggtgcaaatttgtctccactccgtggctgaattcgactaactccgtagaatcgaacagtgcgtgtcgtgtaccaatcctg

SEQ ID NO: 2 - protein, 462 aa, Oryza sativa
  amino acid sequence of OsSRO1c
MDFSGDVKPAIHRPSVAAARGGGNGGAIPLLRGWQAFRRSGAPARLLCFEGGAWADVAGEVVGLLRRAFMEGKAVCEAACGGRVFLFDFMRMVRIDEATAEEAALGWIDDRGACFFPAPEGGRKRKRERDAGSEVKGEDRRRRQPAAEEEDGDEASSGVEERSGESRPEADEPDRKKARGTLWGKAVRLDEADKFYKVVEKLFVSRMAPVAAARGVAITAVHKVAQGPRARAFHLQGQLLAAARGVGDGSNAKFAWYGAPAADVAAAVEHGFGRTNGQFLGGRAHGDGVHLSPPQYPHASAMLTKPDENGEAHIVLCRVLMGRPEAVPASSPQFHPSSDEYDSAVDNLENPRWYVVWSTDMNTRILPEYVVSFRWPNLPQMEGSSGLGSKLKKPSPAATRDMFPMLLTEIQRFVPSPKLQTLQRTYNCFKRGQMKKDQFIRFLRSHIGDNVLTTVAKKLRGY

SEQ ID NO: 3 - DNA, 17 bp, Artificial Sequence
  OsSRO1c-2F primer for amplifiying OsSRO1c gene
ctcccacatcggcgaca

SEQ ID NO: 4 - DNA, 22 bp, Artificial Sequence
  OsSRO1c-2R primer for amplifying OsSRO1c gene
accttgcactagtaccctcgga

SEQ ID NO: 5 - DNA, 21 bp, Artificial Sequence
  AF primer for amplifying a 76 bp fragment of the rice Actin1 gene (Accession No. X16280)
tggcatctctcagcacattcc

SEQ ID NO: 6 - DNA, 20 bp, Artificial Sequence
  AR primer for amplifying a 76 bp fragment of the rice Actin1 gene (Accession No. X16280)
tgcacaatggatgggtcaga

SEQ ID NO: 7 - DNA, 901 bp, Artificial Sequence
  sequence flanking the OsSRO1c T-DNA mutant 3A-05508
natcnnngagggnnanancgggacngcatatnacctgcatatacctgtnagaatttagcaccccaggttagccntgtanttggccacatagcngaaaaaatagcaccgcggnantaagaatggnnctcacctggtacctggtacctcggatccgtgtttgagagttgtttgtttctctggattattagaggcatctaagggtagtgggtctttagctactcaataatctagaaaaagcttctctngaggagattattagattatagtaatctaagctcctctngaggagattattatattatagtaatctggcttataaattataataatctaacataataatctacttatttgtttcagcttaatcctaataatccagattataataatcctaagctgaattaaacagggcctaaattcacctagcagatcagcagttgtgnttcagcccattgtttcgcttttacttatccaaacaatttattaaccaaaaaaaggtatagtaaaaccttttttatacgtgttcatgcggtaaaataaacnactaanaaaaccacaaaatcaactcttatattaaattttgacttacaggcatgatcatangcgcgaaggggttgttttgtgtaagccagcgcaagattctnatcaatcatctntctngaatgaaatgctcagcaagaagtaccgtttaccttgaagcagttgtncntcctctgcaaagtctgcngccggtngcggggccatggaaagncaananacngccgnctgnttgaaaccccaaccaacgttctntgaaaccccanccaanttttntnnaancccaaccacttcnggnggnnccngttcnntnnacgggggggnnganncattaagagggggnngggnnttnaagggggggggn

SEQ ID NO: 8 - DNA, 22 bp, Artificial Sequence
  OsSRO1c-1F primer for amplifying OsSRO1c gene
tccctatgcttctgacggagat

SEQ ID NO: 9 - DNA, 24 bp, Artificial Sequence
  OsSRO1c-1R primer for amplifying OsSRO1c gene
cagttgtacgtcctctgcaaagtc

SEQ ID NO: 10 - DNA, 25 bp, Artificial Sequence
  OsSRO1c FLF - sequence specific primer with one KpnI cleavage site
cagggtaccgggaggggtgatggac

SEQ ID NO: 11 - DNA, 30 bp, Artificial Sequence
  OsSRO1c FLR - sequence specific primer with one KpnI cleavage site
cagggtaccactatgaccgaactcaagaat

(19) United States
(12) Patent Application Publication
(10) Pub. No.: US 2014/0208456 A1
(43) Pub. Date: Jul. 24, 2014

(54) Application of OsSRO1c Gene in Controlling Rice Drought Resistance

(71) Applicants: Lizhong Xiong, Wuhan (CN); Jun You, Wuhan (CN)

(72) Inventors: Lizhong Xiong, Wuhan (CN); Jun You, Wuhan (CN)

(73) Assignee: HUAZHONG AGRICULTURAL UNIVERSITY, Wuhan (CN)

(21) Appl. No.: 14/009,373

(22) PCT Filed: Apr. 1, 2012
(86) PCT No.: PCT/CN2012/073471

§ 371 Date: Feb. 21, 2014

(30) Foreign Application Priority Data
Apr. 2, 2011 (CN) 201110083226.9

(51) Int. Cl. C12N 15/82

(52) U.S. Cl. CPC C12N 15/8273
USPC 800/278; 435/320.1; 800/298; 435/419; 800/320.1; 800/314; 800/312; 800/320.2; 435/412; 435/415; 800/320.3; 47/58.1 R

(57) ABSTRACT

Rice OsSRO1C gene conferring enhanced drought resistance of a plant is provided, wherein the said gene encodes an amino acid sequences as set forth in SEQ ID NO: 2. DNA constructs, transgenic plants or cells comprising the OsSRO1C gene and the uses of the OsSRO1C gene in improving though resistance of a plant are also provided. The OsSRO1C gene controlling rice drought resistance is cloned by screening a rice T-DNA insertion mutant database and determination of expression level and identification of drought stress phenotype, which shows that the mutant is co-segregated with the drought susceptible phenotype.

### TECHNICAL FIELD

The present invention relates to the field of rice genetic engineering, in particular to obtaining of rice OsSRO1c gene conferring enhanced drought resistance by isolation, cloning and functional conformation and the use thereof in genetic improvement office drought resistance. In the present invention, the OsSRO1c gene controlling rice drought resistance was cloned by using candidate gene screening method and the co-segregation detection shows that OsSRO1c mutant is closely related with drought susceptible phenotype. In addition, the overexpression of OsSRO1c gene can improve the drought resistance of transgenic rice, showing the function of said gene and the use thereof.

### BACKGROUND ART

Plants usually are affected by many environmental factors during growth period, wherein drought, coldness and high temperature may result in great reduction of crop production, which is the bottleneck of agricultural development in many areas. Thus, it always a major object to develop crop species with stress tolerance in researches of agricultural science and technology. For resisting or adapting to adverse environmental factors, plants sense extracellular changes of environmental conditions and transfer them through various pathways to the inside of cells to induce expressions of some responding genes and generate some functional proteins, osmoregulation substances as well as transcription factors responsible for signal transmission and gene expression regulation so that plants are able to make corresponding responses to environmental changes and avoid damages caused by stresses such as drought, high salinity and coldness (Xiong et al, Cell signaling during cold, drought and salt stress. Plant Cell. 14 (suppl), S165-S183, 2002). Whether these functional genes for responding to environmental changes can be correctly expressed is finely regulated by some regulatory factors. When plants sense environmental stresses, the transcription factors as regulatory genes are able to regulate the expression of a series of downstream genes to enhance the tolerance of plants to the undesired stresses. Most kinds of the transcription factors are involved in the abiotic stress response, including AP2/EREBP, bZip, MYB, MYC, NAC and Zinc finger like transcription factors (Yamaguchi-Shinozaki K, Shinozaki K. Transcriptional regulatory networks in cellular responses and tolerance to dehydration and cold stresses. Annu Rev Plant Biol, 2006, 57:781-803). By genetic engineering, several stress responsive transcription factors have been successfully applied in breeding stress resistant rice. The seed setting of the SNAC1 transgenic rice strains can be enhanced by about 30% in the field under drought stress conditions while showing no yield or phenotypic changes under normal condition.

The transgenic strains also shows significantly improved drought resistance and salt tolerance at the vegetative stage (Hu et al. Overexpressing a NAM, ATAF, and CUC (NAC) transcription factor enhances drought resistance and salt tolerance in rice. Proc Natl Acad Sci USA, 2006, 103: 12987-12992). These stress responsive transcription factors perform their function by regulating the expression of many downstream genes, among which there are typically some proteins involved in the regulation of signal transduction and gene expression, and thereby secondary regulatory network forms. Some of these downstream genes might also be used in genetic improvement of crop stress resistance. Downstream gene HsfA3 of heat resistant transcription factor DREB2A in Arabidopsis thaliana can improve the heat resistance in the transgenic and overexpression plant (Yoshida et al. Functional analysis of an Arabidopsis heat-shock transcription factor HsfA3 in the transcriptional cascade downstream of the DREB2A stress-regulatory system. Biochem Biophys Res Commun, 2008, 368: 515-21).

SRO (SIMILAR-TO-RCD-ONE) protein family is a class of plant specific proteins identified recently and all the members thereof contain Poly (ADP-ribose) polymerase catalytic domain (PARP) and RCD1-SRO-TAF4 (RST) domain. The research of Arabidopsis thaliana SRO protein RCDI shows that said family member is involved in the responses to oxidation stress and high salt stress and signal transduction of abscisic acid, jasmonic acid and ethylene, and meanwhile affects the development-related phenotypes such as leaf shape and earlier flowering (Ahlfors R et al. Arabidopsis RADICAL-INDUCED CELL DEATH1 belongs to the WWE protein-protein interaction domain protein family and modulates abscisic acid, ethylene, and methyl jasmonate responses. Plant Cell, 2004, 16: 1925-1937). Furthermore, the interactions between RCD1 and many transcription factors affect the expression of a plurality of downstream genes, resulting in the various phenotypes (Jaspers et al. Unequally redundant RCD1 and SRO 1 mediate stress and developmental responses and interact with transcription factors. Plant J, 2009, 60: 268-279). There are 5 SRO family members in rice, however, their functions have not been reported till now. OsSRO1c gene of the present invention is a member of rice SRO family and also be the downstream target gene regulated by SNAC 1.

Rice is one of the most important alimentary corps and model plants. In recent years with frequent appearance of the extreme weathers, breeding for rice with increased tolerance to drought is of particular importance. OsSRO1c gene is one of the downstream genes of the drought resistant transcription factor SNAC1, there has been no relevant report as to whether said gene could improve rice stress resistance till now.

### SUMMARY OF THE INVENTION

One object of the invention relates to the application of OsSRO1c gene, a member of SRO family in rice, in improving drought resistance of a plant, especially of a rice plant. One of the genes with increased expression in the SNAC 1 overexpression plant was selected and the applicant named the gene of the invention as OsSRO1c as it is a member of SRO family. The present invention has isolated and used a DNA fragment containing OsSRO1c gene, the nucleotide sequence of which is set forth in SEQ ID NO: 1 with 1550 bp in length. The coding sequence of the OsSRO1c gene correspond to nt position 77 to nt position 1465 of SEQ ID NO.1 and the corresponding amino acid sequence is set forth in SEQ ID NO: 2 with 462 amino acids in length. It has been demonstrated in the present invention that the rice OsSRO1c gene of the present invention confers increased resistance to drought stress in plants such as rice.

Therefore, the present invention relates to the following embodiments among others:

Item 1. Use of OsSRO1c gene controlling drought resistance in genetically improving drought resistance of a plant, wherein said gene encodes an amino acid sequence as set forth in SEQ ID NO: 2.

Item 2. The use according to Item 1, wherein said gene has a coding sequence as shown from nt position 77 to nt position 1465 of SEQ ID NO. 1.

Item 3. The use according to Item 1, wherein said gene has a nucleotide sequence as set forth in SEQ ID NO.1.

Item 4. A method of improving drought resistance of a plant, wherein said plant is subjected to a treatment so that OsSRO1c gene is expressed at an increased level in said plant in comparison with an identical control plant without the treatment, wherein said gene encodes an amino acid sequence as set forth in SEQ ID NO: 2.

Item 5. The method according to Item 4, wherein said gene has a coding sequence as shown from nt position 77 to nt position 1465 of SEQ ID NO. 1.

Item 6. The method according to Item 4, wherein said gene has a nucleotide sequence as set forth in SEQ ID NO.1.

Item 7. The method according to any one of Items 4-6, wherein said treatment is transformation of said plant by a DNA construct comprising said gene.

Item 8. A DNA construct comprising OsSRO1c gene, wherein said gene encodes an amino acid sequence as set forth in SEQ ID NO: 2.

Item 9. The DNA construct according to Item 8, wherein said gene has a coding sequence as shown from nt position 77 to nt position 1465 of SEQ ID NO. 1.

Item 10. The DNA construct according to Item 8, wherein said gene has a nucleotide sequence as set forth in SEQ ID NO.1.

Item 11. A transgenic plant or a cell thereof comprising transformed OsSRO1c gene in its genome, wherein said gene encodes an amino acid sequence as set forth in SEQ ID NO: 2.

Item 12. The transgenic plant or cell according to Item 11, wherein said gene has a coding sequence as shown from nt position 77 to nt position 1465 of SEQ ID NO. 1.

Item 13. The transgenic plant or cell according to Item 11, wherein said gene has a nucleotide sequence as set forth in SEQ ID NO.1.

In any one of the above Items 1-13, said plant is selected from the group consisting of corn, cotton, soybean, rice and wheat plants, preferably said plant is rice.

### DETAILED DESCRIPTION OF THE INVENTION

Unless specifically defined, all technical and scientific terms used herein have the same meaning as commonly understood by persons of ordinary skill in the art. The procedures for preparing and screening transgenic plants described below are well known and commonly employed by persons of ordinary skill in the art.

The OsSRO1c gene of the present invention is useful in genetically improving drought resistance of a plant such as rice. The gene can be engineered into a vector to form a DNA construct. The DNA construct can be used in transformation of a plant cell or tissue of a plant such as rice to produce a transgenic plant cell and/or transgenic plant. In the transgenic plant cell and transgenic plant, the OsSRO1c gene of the present invention is expressed in an increased level which confers increase level of drought resistance. Therefore, the present invention also relates to a method of improving drought resistance of a plant, wherein said plant is subjected to a treatment so that the OsSRO1c gene of the present invention is expressed at an increased level in said plant in comparison with an identical control plant without the treatment. The OsSRO1c gene, DNA construct comprising said gene, plant cells or plants comprising transformed OsSRO1c gene and use and method of use of the gene of the present invention for improving drought resistance of a plant such as rice are all encompassed in the present invention.

A transgenic “plant cell” means a plant cell that is transformed with stably-integrated, non-natural, recombinant polynucleotides, e.g. by Agrobacterium-mediated transformation or by bombardment using micropaiticles coated with recombinant polynucleotides. A plant cell of this invention can be an originally-transformed plant cell that exists as a microorganism or as a progeny plant cell that is regenerated into differentiated tissue, e.g. into a transgenic plant with stably-integrated, non-natural recombinant polynucleotides in its chromosomal DNA, or seed or pollen derived from a progeny transgenic plant.

A “transgenic” plant or seed means one whose genome has been altered by the stable incorporation of recombinant polynucleotides in its chromosomal DNA, e.g. by transformation, by regeneration from a transformed plant from seed or propagule or by breeding with a transformed plant. Thus, transgenic plants include progeny plants of an original plant derived from a transformation process including progeny of breeding transgenic plants with wild type plants or other transgenic plants. The enhancement of a desired trait can be measured by comparing the trait property in a transgenic plant which has recombinant DNA conferring the trait to the trait level in a progenitor plant.

“Gene expression” means the function of a cell to transcribe recombinant DNA to in RNA and translate the mRNA to a protein. For expression the recombinant DNA usually includes regulatory elements including 5′ regulatory elements such as promoters, enhancers, and introns; other elements can include polyadenylation sites, transit peptide DNA, markers and other elements commonly used by those skilled in the art. Promoters can be modulated by proteins such as transcription factors and by intron or enhancer elements linked to the promoter.

“An increased level” of expression means an increase in the gene expression that is helpful for the drought resistance of the plant, e.g., at least about 5%, at least about 10%, at least about 15%, at least about 20%, at least about 25%, at least about 30%, at least about 40% at least about 50%, at least about 100%, at least about 200% increase in comparison with an identical control without the treatment of the present invention.

“Recombinant polynucleotide” means a DNA construct that is made by combination of two otherwise separated segments of DNA, e.g., by chemical synthesis or by the manipulation of isolated segments of nucleic acids by genetic engineering techniques. Recombinant DNA can include exogenous DNA or simply a manipulated native DNA. Recombinant DNA for expressing a protein in a plant is typically provided as an expression cassette which has a promoter that is active in plant cells operably linked to DNA encoding a protein, linked to a 3′ DNA element for providing a polyadenylation site and signal. Useful recombinant DNA also includes expression cassettes for expressing one or more proteins conferring stress tolerance.

Recombinant DNA constructs generally include a 3″ element that typically contains a polyadenylation signal and site. Well-known 3′ elements include those from Agrobacterium tumefaciens genes such as nos 3′, tml 3′, ttnr 3′, tms 3′, ocs 3\ tr73′, e.g., disclosed in U.S. Pat. No. 6,090,627. 3′ elements from plant genes such as a rice glutelin gene, a rice lactate dehydrogenase gene and a rice beta-tubulin gene are disclosed in U.S. published patent application 2002/0192813 A1.

The expression vector carrying the OsSRO1c gene of the present invention can be introduced into plant cells with Ti plasmid or plant viral vector using the conventional biological technology methods such as direct DNA transformation, microinjection and electroporation (Weissbach, 1998, Method for Plant Molecular Biology VIII, Academy Press, New York, pp. 411-463; Geiserson and Corey, 1998, Plant Molecular Biology (2nd Edition)).

The expression vectors comprising the OsSRO1c gene of the present invention can be transformed into multiple host plants including rice to breed plant varieties with drought resistance.

### Plant Cell Transformation Methods

Numerous methods for transforming plant cells with recombinant DNA are known in the art and may be used in the present invention. Two commonly used methods for plant transformation are Agrobacterium-mediated transformation and microprojectile bombardment. Microprojectile bombardment methods are illustrated in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn) and U.S. Pat. No. 6,153,812 (wheat) and Agrobacterium-mediated transformation is described in U.S. Pat. No. 5,159,135 (cotton); U.S. Pat. No. 5,824,877 (soybean); U.S. Pat. No. 5,591,616 (corn); and U.S. Pat. No. 6,384,301 (soybean), all of which are incorporated herein by reference. For Agrobacterium tumefaciens based plant transformation system, additional elements present on transformation constructs will include T-DNA left and right border sequences to facilitate incorporation of the recombinant polynucleotide into the plant genome.

In general it is useful to introduce recombinant DNA randomly, i.e. at a non-specific location, in the genome of a target plant line. In special cases it may be useful to target recombinant DNA insertion in order to achieve site-specific integration, for example to replace an existing gene in the genome, to use an existing promoter in the plant genome, or to insert a recombinant polynucleotide at a predetermined site known to be active for gene expression. Several site specific recombination systems exist which are known to function implants include cre-lox as disclosed in U.S. Pat. No. 4,959,317 and FLP-FRT as disclosed in U.S. Pat. No. 5,527,695.

Transformation methods of this invention are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Cells capable of proliferating as callus are also recipient cells for genetic transformation. Practical transformation methods and materials for making transgenic plants of this invention, for example various media and recipient target cells, transformation of immature embryo cells and subsequent regeneration of fertile transgenic plants are disclosed in U.S. Pat. Nos. 6,194,636 and 6,232,526, which are incorporated herein by reference.

The seeds of transgenic plants can be harvested from fertile transgenic plants and be used to grow progeny generations of transformed plants of this invention including hybrid plants line for selection of plants having an enhanced trait. In addition to direct transformation of a plant with a recombinant DNA, transgenic plants can be prepared by crossing a first plant having a recombinant DNA with a second plant lacking the DNA. For example, recombinant DNA can be introduced into first plant line that is amenable to transformation to produce a transgenic plant which can be crossed with a second plant line to introgress the recombinant DNA into the second plant line. A transgenic plant with recombinant DNA providing an enhanced trait, e.g. enhanced yield, can be crossed with transgenic plant line having other recombinant DNA that confers another trait, for example drough resistance or pest resistance, to produce progeny plants having recombinant DNA that confers both traits.

In the practice of transformation DNA is typically introduced into only a small percentage of target plant cells in any one transformation experiment. Marker genes are used to provide an efficient system for identification of those cells that are stably transformed by receiving and integrating a transgenic DNA construct into their genomes. Preferred marker genes provide selective markers which confer resistance to a selective agent, such as an antibiotic or herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin and paromomycin (npt11), hygromycin B (aph IV) and gentamycin (aac3 and aacCA) or resistance to herbicides such as glufosinate {bar or pat) and glyphosate (aroA or EPSPS). Examples of such selectable are illustrated in U.S. Pat. Nos. 5,550,318; 5,633,435; 5,780,708 and 6,118,047. Selectable markers which provide an ability to visually identify transformants can also be employed, for example, a gene expressing a colored or fluorescent protein such as a luciferase or green fluorescent protein (GFP) or a gene expressing a fteto-glucuronidase or uidA gene (GUS) for which various chromogenic substrates are known.

Plant cells that survive exposure to the selective agent, or plant cells that have been scored positive in a screening assay, may be cultured in regeneration media and allowed to mature into plants. Developing plantlets regenerated from transformed plant cells can be transferred to plant growth mix, and hardened off, for example, in an environmentally controlled chamber at about 85% relative humidity, 600 ppm CO2, and 25-250 microeinsteins m⁻¹s⁻¹ of light, prior to transfer to a greenhouse or growth chamber for maturation. Plants are regenerated from about 6 weeks to 10 months after a transformant is identified, depending on the initial tissue. Plants may be pollinated using conventional plant breeding methods known to those of skill in the art and seed produced, for example self-pollination is commonly used with transgenic corn. The regenerated transformed plant or its progeny seed or plants can be tested for expression of the recombinant DNA and selected for the presence of enhanced agronomic trait.

### Transgenic Plants and Seeds

Transgenic plants derived from the plant cells of this invention are grown to generate transgenic plants having an enhanced trait as compared to a control plant and produce transgenic seed and haploid pollen of this invention. Such plants with enhanced traits are identified by selection of transformed plants or progeny seed for the enhanced trait.

For efficiency a selection method is designed to evaluate multiple transgenic plants (events) including the recombinant DNA, for example multiple plants from 2 to 20 or more transgenic events. Transgenic plants grown from transgenic seed provided herein demonstrate improved agronomic traits that contribute to increased yield or enhanced water deficit tolerance or both.

Not all transgenic events will be in transgenic plant cells that provide plants and seeds with an enhanced or desired trait depending on factors, such as location and integrity of the recombinant DNA, copy number, unintended insertion of other DNA, etc. As a result transgenic plant cells of this invention are identified by screening transformed progeny plants for enhanced water deficit stress tolerance and yield. For efficiency a screening program is designed to evaluate multiple transgenic plants preferably with a single copy of the recombinant DNA from 2 or more transgenic events.

Since the expression of the gene of the present invention is induced by drought stress, the gene of the present invention can be inserted into suitable expression vector with combination with any drought inducible promoter of interest and then transformed into plant hosts, wherein the expression of said gene thereby can be induced under drought condition, enhancing the drought resistance of the transgenic plant thereof.

The further illustration of the invention is given below in reference to the figures and examples.

### BRIEF DESCRIPTION OF THE FIGURES

SEQ ID NO:1 is the nucleotide acid sequence containing the coding region of the OsSRO1c gene isolated and cloned in the invention with 1550 bp in length, and the amino acid sequence of the corresponding protein thereof is set forth in SEQ ID NO: 2 with 463 amino acids in length.

FIG. 1. Expression pattern of OsSRO1c gene under various adverse situations and hormone treatments. The samples were treated as follows: drought treatment for 0 d, 3 d, 5 d and 7 d; High salt treatment for 0 h, 3 h, 6 h and 12 h; Cold treatment for 0 h, 6 h, 12 h and 24 h; Heat treatment for 0 min, 10 min, 30 min and 2 h; UV-C treatment for 0 h, 3 h, 6 h and 12 h; Wound treatment for 0 h, 1 h, 3 h and 6 h; H₂O₂ stress treatment for 0 h, 2 h, 6 h and 12 h; Submerge treatment for 0 h, 6 h, 24 h and 72 h. Hormone treatments: treatment with abscisic acid (ABA), brassinosteroid (BR), indoleacetic acid (IAA), kinetin(KT), gibberellic acid (GA), jasmonic acid (JA), salicylic acid (SA) and ethephon (ETH) for 0 h, 2 h, 6 h and 12 h.

FIG. 2. Expression pattern of OsSRO1c gene in OsSRO1c mutant. Two short red lines (a, b) represent the two sites corresponding to primer pair flanking the insertion site for T-DNA. WT is the negative control isolated from transgenic lines and osrsol is the T-DNA insertion mutant.

FIG. 3 Drought stress phenotypes of ossro1c mutant rice at seedling stage. Ossro1c#1, #2 and #3 are three T-DNA insertion mutant lines and WT#1, #2 and #3 are three negative lines isolated from heterozygous lines.

FIG. 4. Survival rate statistics of ossro1c mutant rice at seedling stage under drought stress. Ossro1c#1, #2 and #3 are three mutant lines and WT#1, #2 and #3 are three negative lines isolated from heterozygous lines.

FIG. 5. Drought stress phenotype of ossro1c mutant rice at adult plant stage under field drought stress. Ossro1c is the homozygous mutant and WT is isolated negative lines.

FIG. 6. Biomass statistics of ossro1c mutant rice at adult plant stage under field drought stress. Ossro1c is the homozygous mutant and WT is isolated negative lines.

FIG. 7. Expression pattern of OsSRO1c gene in OsSRO1c-OX overexpression plant lines. Zhonghua 11 (ZH11) is a wild type line.

FIG. 8. Osmosis responsive phenotype of the OsSRO1c-OX overexpression plant line. OsSRO1c-OX-1, -11 and -14 are three independent overexpression transgenic T1 lines and Zhonghua 11 (ZH11) is a wild type line.

FIG. 9. Height statistics of the OsbZIP46CA1 overexpression plant lines under osmosis stress. OsSRO1c-OX-1, -11 and -14 are three independent overexpression transgenic T1 lines and Zhonghua 11 (ZH11) is a wild type line.

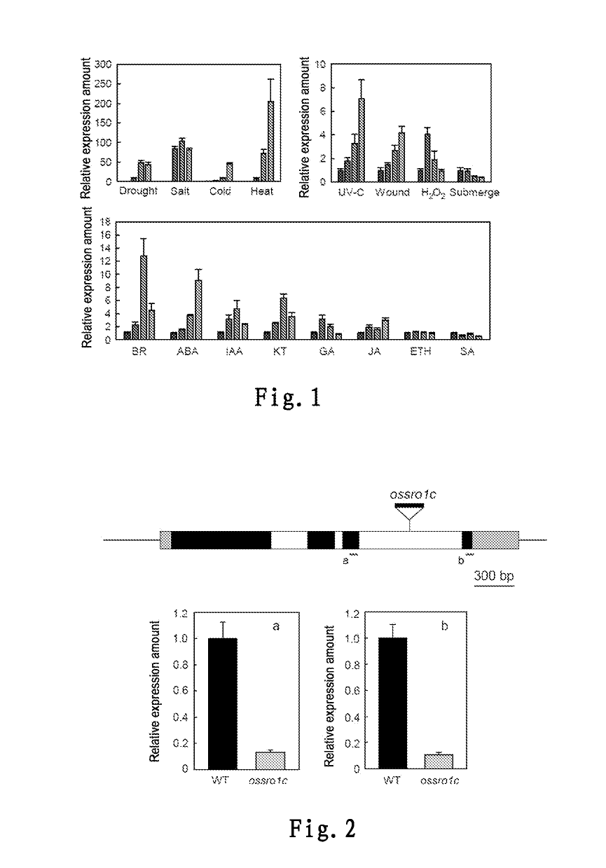

The following examples are included to demonstrate embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments which are disclosed and still obtain a like or similar result without departing from the spirit and scope of the invention, therefore all matter set forth or shown in the accompanying drawings and examples is to be interpreted as illustrative and not in a limiting sense.

### EXAMPLES

The following examples describe methods for isolating OsSRO1c T-DNA insertion mutant and cloning the DNA fragment comprising the entire encoding region of OsSRO1c gene and for verifying the function of OsSRO1c gene.

### Example 1

### Determination of the Expression Level of the Endogenous Rice OsSRO1c Gene Induced by Stress

To preliminarily judge whether the OsSRO1c gene is related to stress response, the applicant firstly determined whether the expression level of the endogenous rice OsSRO1c gene is induced under stress conditions. The applicant selected rice indica “Zhonghua 11” (ZH11, commercial line from Crop Research Institute, Chinese Agriculture Academy) as the line for analyzing the expression pattern. The 4-leaf seedlings were treated under various stress conditions or hormones. The drought treatment was conducted by naturally drying without watering for 0 h, 3 h, 5 h, 7 h, and then sampling. The high-salinity treatment was conducted by immersing the seedling root in 200 mM NaCl solution for 0 h, 3 h, 6 h, 8 h, and then sampling. The coldness treatment was conducted by placing the seedling in a 4° C. growth chamber for 0 h, 6 h, 12 h, 24 h, and then sampling. The heat treatment was conducted by placing the seedling in a 42° C. growth chamber for 0 min, 10 min, 30 min, 2 h, and then sampling. The ultraviolet ray treatment was conducted by placing the seedling under ultraviolet lamp for 0 h, 3 h, 6 h, 12 h, and then sampling. The wound treatment was conducted by mechanically injuring the seedling with forceps and then sampling after 0 h, 1 h, 3 h and 6 h. The oxidation treatment was conducted by immersing the seedling root in 1% H₂O₂ solution for 0 h, 2 h, 6 h, 8 h, and then sampling. The submerge treatment was conducted by placing the seedling in transparent chamber filled with water for 0 h, 6 h, 12 h, 24 h, and then sampling. Hormone treatment was conducted by spraying each of abscisic acid (ABA), brassinosteroid (BR), indoleacetic acid (IAA), kinetin (KT), gibberellic acid (GA), jasmonic acid (JA), salicylic acid (SA) and ethephon (ETH) to the surface of the rice plant and root of the seedling and sampling after 0 h, 2 h, 6 h and 12 h. Total RNA was extracted using TRIZOL reagent (from Invitrogen Co.) according to the specification of the manufacturer and reverse transcribed to cDNA using reverse transcriptase SSIII (from Invitrogen Co.) according to the specification of the manufacturer. The reaction was conducted as follows: 65° C. 5 min, 50° C. 120 min, 70° C. 10 min. With the above reverse transcribed cDNA as template, OsSRO1c gene was specifically PCR amplified using primers (OsSRO1c-2F: 5′-CTCCCACATCGGCGACA-3′ and OsSRO1c-2R: 5′-ACCTTGCACTAGTACCCTCGGA-3′). Meanwhile, a 76 bp fragment of the rice Actin1 gene (Accession No. X16280) was specifically amplified with primers (AF: 5′-TGGCATCTCTCAGCACATTCC-3′ and AR: 5′-TGCACAAT GGATGGGTCAGA-3′) as internal control for quantitative analysis. PCR reaction was conducted as follows: 95° C. 10 sec; 95° C. 5 sec, 60° C. 34 sec, 40 cycles. Fluorescent real time quantitative analysis was conducted during the reaction process. The results demonstrate that the expression level of OsSRO1c gene (SEQ NO: 1) is enhanced under drought, high-salinity, coldness, heat, ultraviolet rays, injury and oxidation stress and decreased under submerge stress. Moreover, the expression level of OsSRO1c gene is enhanced after induction by several hormones such as ABA, BR, IAA, KT, GA and JA.

### Example 2

### Isolation of OsSRO1c Gene Mutant

For identifying the stress resistance capability of OsSRO1c gene, one critical step is to obtain the mutant thereof. The T-DNA insertion mutant 3A-05508 of OsSRO1c gene was selected from Rice T-DNA Insertion Sequence Database(RISD) (The starting material of the present invention is mutant 3A-05508, the website: http://signal.salk.edu/cgi-bin/RiceGE, POSTECH Plant Functional Genomics Laboratory of Korea). The methods of constructing this mutant vector and genetic transformation are described in the related literatures (Jeong et al., Generation of a flanking sequence-tag database for activation-tagging lines in japonica rice. Plant J. 2006, 45:123-32.) and the present description does not provide more description in detail. In the above mentioned mutant database, the sequence (with 901 bp in length) flanking the OsSRO1c T-DNA mutant 3A-05508 is set forth as follows:

NATCNNNGAGGGNNANANCGGGACNGCATATNACCTGCATATACCTGTNA
GAATTTAGCACCCCAGGTTAGCCNTGTANTTGGCCACATAGCNGAAAAAA
TAGCACCGCGGNANTAAGAATGGNNCTCACCTGGTACCTGGTACCTCGGA
TCCGTGTTTGAGAGTTGTTTGTTTCTCTGGATTATTAGAGGCATCTAAGG
GTAGTGGGTCTTTAGCTACTCAATAATCTAGAAAAAGCTTCTCTNGAGGA
GATTATTAGATTATAGTAATCTAAGCTCCTCTNGAGGAGATTATTATATT
ATAGTAATCTGGCTTATAAATTATAATAATCTAACATAATAATCTACTTA
TTTGTTTCAGCTTAATCCTAATAATCCAGATTATAATAATCCTAAGCTGA
ATTAAACAGGGCCTAAATTCACCTAGCAGATCAGCAGTTGTGNTTCAGCC
CATTGTTTCGCTTTTACTTATCCAAACAATTTATTAACCAAAAAAAGGTA
TAGTAAAACCTTTTTTATACGTGTTCATGCGGTAAAATAAACNACTAANA
AAACCACAAAATCAACTCTTATATTAAATTTTGACTTACAGGCATGATCA
TANGCGCGAAGGGGTTGTTTTGTGTAAGCCAGCGCAAGATTCTNATCAAT
CATCTNTCTNGAATGAAATGCTCAGCAAGAAGTACCGTTTACCTTGAAGC
AGTTGTNCNTCCTCTGCAAAGTCTGCNGCCGGTNGCGGGGCCATGGAAAG
NCAANANACNGCCGNCTGNTTGAAACCCCAACCAACGTTCTNTGAAACCC
CANCCAANTTTTNTNNAANCCCAACCACTTCNGGNGGNNCCNGTTCNNTN
NACGGGGGGGNNGANNCATTAAGAGGGGGNNGGGNNTTNAAGGGGGGGGG
N.
(Note:
N means undetermined nucleotide in sequencing.)

Primers were designed according to the insertion site of T-DNA and the expression amount of OsSRO1c gene in the mutant was determined. OsSRO1c gene was specifically PCR amplified with the primers (OsSRO1c-1F: 5′-TCCCTATGCTTCTGACGGAGAT-3′ and OsSRO1c-1R: 5′-CAGTTGTACGTCCTCTGCAAAGTC-3′) and (OsSRO1c-2F: 5′-CTCCCACATCGGCGACA-3′ and OsSRO1c-2R: 5′-ACCTTGCACTAGTACCCTCGGA-3′). Meanwhile, a 76 bp fragment of the rice Actin1 gene was specifically amplified with primers (AF: 5′-TGGCATCTCTCAGCACATTCC-3′ and AR: 5′-TGCACAAT GGATGGGTCAGA-3′) as internal control for quantitative analysis. PCR reaction was conducted as follows: 95° C. 10 sec; 95° C. 5 sec, 60° C. 34 sec, 40 cycles. Real time quantitative analysis with fluorescence detection was conducted during the reaction process. The expression results shows that in the homozygous OsSRO1c T-DNA insertion mutant, the expression level of OsSRO1c gene is significantly lower than that in the isolated negative control (see FIG. 2), indicating that the expression of OsSRO1c gene is significantly inhibited in the mutant.

### Example 3

### Identification of the Drought Stress Phenotype of the Mutant

The sprouting seeds of the homozygous mutant (OsSRO1c) and wildtype line (WT) were seeded in small round buckets. The soil used in the experiments was a mixture of South China rice soil and sands in a ratio of 2:3, the same amount of homologous soil with the same volume of water was added in each bucket and the water naturally leaked out to ensure the consistency of soil compactness. The experiment was in triplet. The healthy plants in 4-leaf stage were treated with drought stress for 6-10 days (depending on the particular weather), then rewatered for 5-7 days and finally the survival rates of the plants were observed. Compared with the wild type control, T-DNA homozygous plants showed the drought susceptible phenotype.

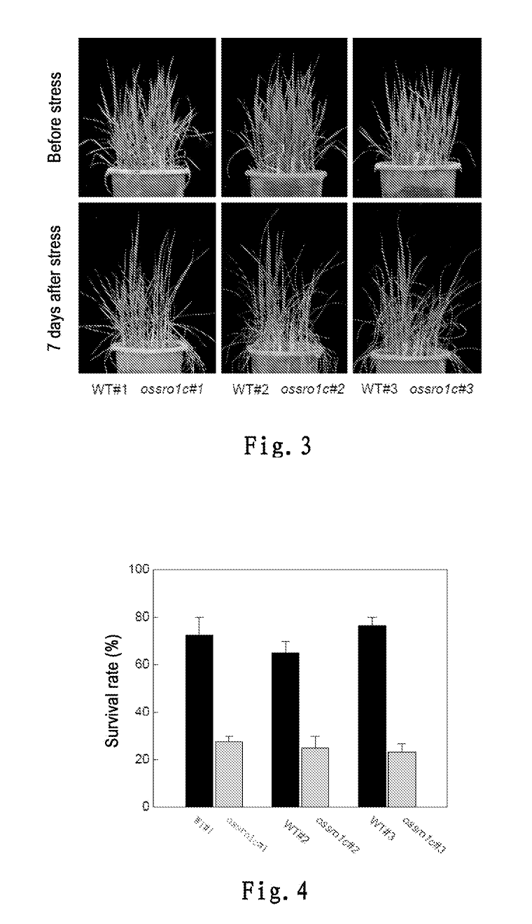

For verifying the co-segregation between said mutant and drought stress phenotype, the homozygous T2 line derived from cultivating seeds of the heterozygous T1 line was crossed with negative seeds to reproduce a new line. Three homozygous mutant lines (OsSRO1c#1, #2, #3) and three isolated wild type lines (WT#1, #2, #3) were randomly selected and subject to the above stress experiments. The results show that the homozygous mutants are more susceptible to drought stress than the negative controls (FIG. 3). After rewatering, the survival rates of the homozygous lines are lower than 30%, while the survival rates of the wild type lines are higher than 60% (FIG. 4). The experiments were in biological triplet and the results are consistent, showing that said mutant phenotype is indeed the result of T-DNA insertion.

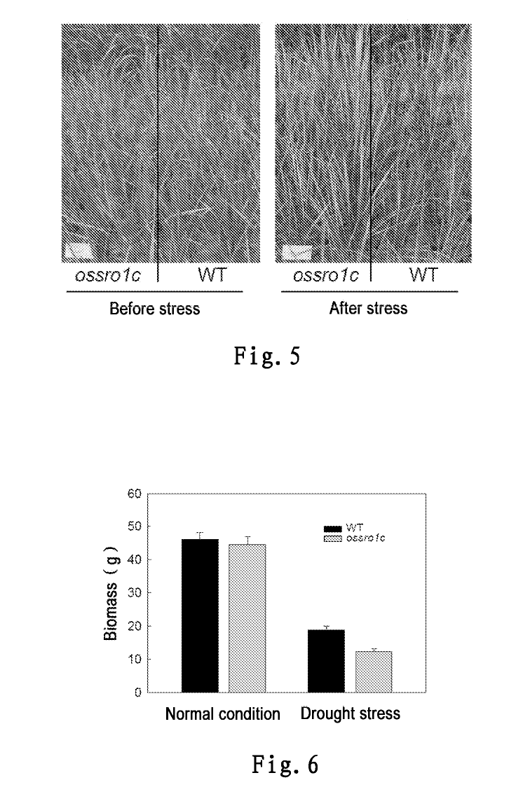

To identify the phenotype of the mutant in the adult plant stage, the mutant lines and the control lines thereof were transplanted to a sandy field above which is provided with a removable canopy, wherein South China rice soil and sands were mixed in a ratio of 1:2 and 10 individual plants of each row and 2 rows for each line were planted. The severe drought stress experiments were in triplet. Drought treatment was conducted by stopping the water supply to the healthy plants in adult plant stage for 15-20 days (depending on the particular weather, be covered with the removable canopy when raining), and then rewatering. Compared with the negative control lines isolated from the heterozygous lines, the leaves of the homozygous mutant plants began to roll earlier, exhibiting drought susceptible phenotype (FIG. 5). Furthermore, the biomass above the ground was harvested and weighed, with the results showing that the biomass of the mutant lines at adult plant stage under drought stress is remarkably lower than the control lines (FIG. 6).

### Example 4

### Construction and Genetic Transformation of the Overexpression Vector of OsSRO1c Gene

In order to further illustrate the stress resistant function of OsSRO1c gene, it was overexpressed in rice and investigated by verifying the phenotype of transgenic plants thereof.

The overexpression vector was constructed as follows: firstly, by searching in two databases, the Annotation No. of OsSRO1c gene is LOC_Os03g12820 and AK102303 in Rice Genome Annotation Project-RGAP(http://rice.plantbio/ogy.msu.edu/) and KOME (http://cdna01.dna.affrc.go.jp/cDNA/) respectively, which is predicted to be a member of SRO family, whose complete nucleotide sequence is set forth as SEQ ID NO: 1, and whose coding region is 1389 bp in length and the corresponding amino acid sequence is 462 amino acids in length, and based on which the primers were designed. A cDNA clone (Accession Number: CT857428) comprising partial sequence of 5′ coding region of OsSRO1c gene is identified by searching in Rice indica cDNA Database (http://www.ncgr.ac.cn/ricd). With this clone as template, using primers OSSRO1CFLF(5′-CAGGGTACCGGGAGGGGTGATGGAC-3′, sequence specific primer with one KpnI cleavage site) and OSSRO1CFLR(5′-CAGGGTACCACTATGACCGAACTCAAGAAT-3′, sequence specific primer with one KpnI cleavage site), a cDNA fragment comprising the complete coding region of OsSRO1c gene was amplified, which corresponds to the sequence 1-1550 bp of the present invention. PCR reaction was conducted as follows: predenaturation at 94° C. for 3 min; 94° C. for 30 sec, 55° C. for 30 sec, 72° C. for 3 min, 30 cycles; and elongation at 72° C. for 5 min. The PCR product was linked into pGEM-T vector (from Promega Co. see Registration Table of Source of Genetic Resource) and the positive clone was screened and sequenced, resulting in the desired full length gene. The clone was named as PGEM-OsSRO1c. Then, enzymatically cleave the positive clone PGEM-OsSRO1c plasmid with KpnI, and recover exogenous fragments, meanwhile, enzymatically cleave the genetic transformation vector pCAMBIA1301U with the ubiquitin promoter in the same way (pCAMBIA1301U was reconstructed based on genetic transformation vector pCAMBIA1301 common used internationally, an Agrobacterium mediated vegetable genetic transformation vector carrying corn ubiquitin promoter with constitutive and over expression characteristics); after cleavage, extract and purify the products of enzymatical cleavage with chloroform: iso-pentanol (24:1 v/v). Conduct linkage reaction between the digested OsSRO1c fragment and digested pCAMBIA1301U vector and then transform the same into E. coli DH10β (the E. coli DH10β strain was purchased from Promega Co.). Identify the positive clone by enzymatic cleavage and the obtained recombination vector was named as OsSRO1c-OX-p1301U (wherein the nucleotide sequence of OsSRO1c gene is set forth in SEQ ID NO: 1 with 1550 bp in length).

By using the Agrobacterium mediated rice genetic transformation method, the above overexpression vector OsSRO1c-OX-p1301U was introduced into the rice variety “Zhong Hua 11”, and a transgenic plant was then obtained by precultivation, infestation, co-culture, screening the callus with hygromycin resistance, differentiation, rooting, seedling training and transplanting. The above Agrobacterium mediated rice (Zhonghua 11) genetic transformation method (system) was resulted from modification on the method reported by Hiei, et al. (Hiei, et al., Efficient transformation of rice, Oryza sativa L., mediated by Agrobacterium and sequence analysis of the boundaries of the T-DNA, Plant J, 6:271-282, 1994).

### The Genetic Transformation Protocol of the Present Example:

(1) Electro formation: Electrotransform the plasmid OsSRO1c-OX-p1301U overexpressing target gene into Agrobacterium strain EHA105 under 1800V, plate the electrotransformation mixture on LA medium and then screen the positive clones to be used in the following callus transformation.
(2) Callus Induction: Mature rice seeds of ZHONGHUA 11 (one public rice line provided by China National Rice Research Institute) were deshelled, then treated with 70% alcohol for 1 minute and disinfected on the surface of the seeds with 0.15% HgCl₂ for 15 minutes. The seeds were washed with sterilized water for 4-5 times. The sterilized seeds were put on the induction medium (see below for components). The inoculated callus induction medium was placed in darkness and cultured for 4 weeks at 25±1° C.
(3) Callus Subculture: The bright yellow, compact and relatively dry embryogenic callus was selected, put onto the subculture medium (see below for components), and cultured in darkness for 2 weeks at 25±1° C.
(4) Pre-Culture: The compact and relatively dry rice embryogenic callus was selected, put onto the pre-culture medium (see below for components), and cultured in darkness for 2 weeks at 25±1° C.
(5) Agrobacterium Culture: Agrobacterium EHA105 (from CAMBIA, commercial strain, carrying the overexpression vector OsSRO1c-OX-p1301U) was precultured on the selective LA medium (see below for components) at the temperature of 28° C. for 2 days; Said Agrobacterium was transferred into suspension medium (as described below) in swing bed at 28° C. for 2-3 hours.
(6) Agrobacterium Infection: The pre-cultured callus was transferred into a sterilized bottle. The said Agrobacterium suspension was regulated to OD₆₀₀ 0.8-1.0. The rice callus was immersed in the Agrobacterium suspension for 30 minute. The callus was transferred on sterilized filter paper and dried, and then cultured onto the cocultivation medium (see below for components) for 3 days at 19-20° C.
(7) Washing and Selective Culture of Callus: The callus was washed with sterilized water until no agrobacrium was observed; immersed in sterilized water containing 400 ppm carbenicillin (CN) for 30 minutes; transferred on sterilized filter paper and dried; and transferred on the selection medium (see below for components) and selectively cultured for 2-3 times, 2 weeks for each time (The carbenicillin concentration was 400 ppm for the first culture, and 250 ppm for the second and subsequent cultures. The hygromycin concentration was 250 ppm.)
(8) Differentiation: The resistant callus was transferred to the pre-differentiation medium (see below for components) and cultured in darkness for 5-7 weeks. The pre-differentiated callus was then transferred to differentiation culture medium (see below for components) and cultured in lighting (3500 lux) at 26° C.
(9) Rooting: The roots generated during the differentiation were cut off. Then the plant was transferred to the rooting culture medium and cultured in lighting at 26° C. for 2-3 weeks.
(10) Transplantation: The residual medium on roots of the plant was washed off, the seedlings with well-grown roots were transplanted into greenhouse, and moisture was maintained in the first several days.

### The Components and Formulations of the Mediums:

### (1) The Abbreviations of the Reagents and Solutions:

The abbreviations of the plant hormones used in the mediums of the invention are: 6-BA (6-Benzyladenine); CN (Carbenicillin); KT (Kinetin); NAA (Napthalene acetic acid); IAA (Indole-3-acetic acid); 2,4-D (2,4-Dichlorophenoxyacetic acid); AS (Acetosringone); CH (Casein Hydrolysate); HN (Hygromycin B); DMSO (Dimethyl Sulfoxide); N6max (N6 solution with major elements); N6mix (N6 solution with trace elements); MSmax (MS solution with major elements); MSmix (MS solution with trace elements)

### (2) The Formulation of the Main Solutions

1) Preparation of Concentrated Solution of N6 Medium with Major Elements (10× concentrated solution):

| | |
|---|---|
| Potassium nitrate (KNO₃) | 28.3 g |
| Monopotassium phosphate (KH₂PO₄) | 4.0 g |
| Ammonium sulfate ((NH₄)₂SO₄) | 4.63 g |
| Magnesium sulfate (MgSO₄•7H₂O) | 1.85 g |
| Calcium chloride (CaCl₂•2H₂O) | 1.66 g |

Dissolve them one by one and add water to the final volume 1000 ml at room temperature.

2) Preparation of Concentrated Solution of N6 Medium with Trace Elements (10× Concentrated Solution):

| | |
|---|---|
| Potassium iodide (KI) | 0.08 g |
| Boracic acid (H₃BO₃) | 0.16 g |
| Manganese sulfate (MnSO₄•4H₂O) | 0.44 g |
| Zinc sulfate (ZnSO₄•7H₂O) | 0.15 g |

Dissolve and add water to the final volume 1000 ml at room temperature.

3) Preparation of the Ferric Salt (Fe₂EDTA) Stock Solution (100×)

800 ml of Double distilled water was heated to 70° C., 3.73 g of Disodium Ethylene Diamine Tetraacetic Acid (Na2EDTA.2H2O) was added therein. After dissolving completely, the solution was kept in 70° C. water bath for 2 hours and water was added to a final volume 1000 ml and then the solution was kept at 4° C. for use.

4) Preparation of the Vitamin Stock Solution (100×)

| | |
|---|---|
| Nicotinic acid | 0.1 g |
| Vitamin B1(Thiamine HCl) | 0.1 g |
| Vitamin B6 (Pyridoxine HCl) | 0.1 g |
| Glycine | 0.2 g |
| Inositol | 10 g |

Add water to a final volume 1000 ml and then keep the solution at 4° C. for use.

5) Preparation of the Concentrated Solution of MS Medium with Major Elements (10×):

| | |
|---|---|
| Ammonium nitrate (NH₄NO₃) | 16.5 g |
| Potassium nitrate | 19.0 g |
| Monopotassium phosphate | 1.7 g |
| Magnesium sulfate | 3.7 g |
| Calcium chloride | 4.4 g |

Dissolve the components and add water to the final volume 1000 ml at room temperature.

6) Preparation of the Concentrated Solution of MS Medium with Trace Elements (10×)

| | |
|---|---|
| Potassium iodide | 0.083 g |
| Boracic acid | 0.62 g |
| Manganese sulfate | 0.86 g |
| Sodium Molybdate (Na₂MoO₄•2H₂O) | 0.025 g |
| Copper sulphate (CuSO₄•5H₂O) | 0.0025 g |

Dissolve the components and add water to the final volume 1000 ml at room temperature.

7) The 2,4-D Stock Solution, the 6-BA Stock Solution, the Naphthylacetic Acid (NAA) stock solution and the indoleacetic acid (IAA) stock solution: 1 mg/ml.

8) The Glucose Stock Solution: 0.5 g/ml.

9) Preparation of the AS Stock Solution: Weigh 0.392 g of AS, in 10 ml DMSO

### (3) The Formulations of the Mediums for Rice Genetic Transformation

### 1) Induction Medium

| | | |
|---|---|---|
| N6max concentrated solution (10X) | 100 | ml |
| N6mix concentrated solution (100X) | 10 | ml |
| Fe²⁺EDTA stock solution (100X) | 10 | ml |
| Vitamin stock solution (100X) | 10 | ml |
| 2,4-D stock solution | 2.5 | ml |
| Proline | 0.3 | g |
| CH | 0.6 | g |
| Sucrose | 30 | g |
| Phytagel | 3 | g |

Add distilled water to 900 ml, adjust pH to 5.9 with 1N potassium hydroxide, boil the solution, add water to a final volume 1000 ml and distribute the solution into 50 ml-triangular flasks (25 ml/flask) followed by seal and sterilization.

### 2) Subculture Medium

| | | |
|---|---|---|
| N6max concentrated solution (10X) | 100 | ml |
| N6mix concentrated solution (100X) | 10 | ml |
| Fe²⁺EDTA stock solution (100X) | 10 | ml |
| Vitamin stock solution (100X) | 10 | ml |
| 2,4-D stock solution | 2.0 | ml |
| Proline | 0.5 | g |
| CH | 0.6 | g |
| Sucrose | 30 | g |
| Phytagel | 3 | g |

Add distilled water to 900 ml, adjust pH to 5.9 with 1 N potassium hydroxide, boil the solution, add water to a final volume 1000 ml and distribute the solution into 50 ml-triangular flasks (25 ml/flask) followed by seal and sterilization.

### 3) Preculture Medium

| | | |
|---|---|---|
| N6max concentrated solution (10X) | 12.5 | ml |
| N6mix concentrated solution (100X) | 1.25 | ml |
| Fe²⁺EDTA stock solution (100X) | 2.5 | ml |
| Vitamin stock solution (100X) | 2.5 | ml |
| 2,4-D stock solution | 0.75 | ml |
| CH | 0.15 | g |
| Sucrose | 5 | g |
| Agar powder | 1.75 | g |

Add distilled water to 250 ml, adjust pH to 5.6 with 1 N potassium hydroxide followed by seal and sterilization. Prior to use, heat and dissolve the medium, add 5 ml glucose stock solution and 250 μl AS stock solution and distribute the solution into dishes (25 ml/dish).

### 4) Coculture Medium

| | | |
|---|---|---|
| N6max concentrated solution (10X) | 12.5 | ml |
| N6mix concentrated solution (100X) | 1.25 | ml |
| Fe²⁺EDTA stock solution (100X) | 2.5 | ml |
| Vitamin stock solution (100X) | 2.5 | ml |
| 2,4-D stock solution | 0.75 | ml |
| CH | 0.2 | g |
| Sucrose | 5 | g |
| Agar powder | 1.75 | g |

Add distilled water to 250 ml, adjust pH to 5.6 with 1 N potassium hydroxide followed by seal and sterilization. Prior to use, heat and dissolve the medium, add 5 ml glucose stock solution and 250 μl AS stock solution and distribute the solution into dishes (25 ml/dish).

### 5) Suspension Culture Medium

| | | |
|---|---|---|
| N6max concentrated solution (10X) | 5 | ml |
| N6mix concentrated solution (100X) | 0.5 | ml |
| Fe²⁺EDTA stock solution (100X) | 0.5 | ml |
| Vitamin stock solution (100X) | 1 | ml |
| 2,4-D stock solution | 0.2 | ml |
| CH | 0.08 | g |
| Sucrose | 2 | g |

Add distilled water to 100 ml, adjust pH to 5.4 and distribute the solution into two 100 ml-triangular flasks followed by sealing and sterilization. Prior to use, add 1 ml glucose stock solution and 100 μl AS stock solution.

### 6) Selective Culture Medium

| | | |
|---|---|---|
| N6max concentrated solution (10X) | 25 | ml |
| N6mix concentrated solution (100X) | 2.5 | ml |
| Fe²⁺EDTA stock solution (100X) | 2.5 | ml |
| Vitamin stock solution (100X) | 2.5 | ml |
| 2,4-D stock solution | 0.625 | ml |
| CH | 0.15 | g |
| Sucrose | 7.5 | g |
| Agar powder | 1.75 | g |

Add distilled water to 250 ml and adjust to 6.0 followed by sealing and sterilization. Prior to use, dissolve the medium, add 250 μl HN and 400 ppm CN and distribute the medium into dishes (25 ml/dish).

### 7) Predifferentiation Medium

| | | |
|---|---|---|
| N6max concentrated solution (10X) | 25 | ml |
| N6mix concentrated solution (100X) | 2.5 | ml |
| Fe²⁺EDTA stock solution (100X) | 2.5 | ml |
| Vitamin stock solution (100X) | 2.5 | ml |
| 6-BA stock solution | 0.5 | ml |
| KT stock solution | 0.5 | ml |
| NAA stock solution | 50 | μl |
| IAA stock solution | 50 | μl |
| CH | 0.15 | g |
| Sucrose | 7.5 | g |
| Agar powder | 1.75 | g |

Add distilled water to 250 ml, adjust pH to 5.9 with 1N potassium hydroxide followed by seal and sterilization. Prior to use, dissolve the medium, add 250μl HN and 200 ppm CN and distribute the medium into dishes (25 ml/dish).

### 8) Differentiation Medium

| | | |
|---|---|---|
| N6max concentrated solution (10X) | 100 | ml |
| N6mix concentrated solution (100X) | 10 | ml |
| Fe²⁺EDTA stock solution (100X) | 10 | ml |
| Vitamin stock solution (100X) | 10 | ml |
| 6-BA stock solution | 2 | ml |
| KT stock solution | 2 | ml |
| NAA stock solution | 0.2 | ml |
| IAA stock solution | 0.2 | ml |
| CH | 1 | g |
| Sucrose | 30 | g |
| Phytagel | 3 | g |

Add distilled water to 900 ml, adjust pH to 6.0 with 1 N potassium hydroxide, boil the solution, add water to a final volume 1000 ml and distribute the solution into 50 ml-triangular flasks (50 ml/flask) followed by seal and sterilization.

### 9) Rooting Medium

| | | |
|---|---|---|
| MSmax concentrated solution (10X) | 50 | ml |
| MSmix concentrated solution (100X) | 5 | ml |
| Fe²⁺EDTA stock solution (100X) | 5 | ml |
| Vitamin stock solution (100X) | 5 | ml |
| Sucrose | 30 | g |
| Phytagel | 3 | g |

Add distilled water to 900 ml, adjust pH to 5.8 with 1 N potassium hydroxide, boil the solution, add water to a final volume 1000 nil and distribute the medium into rooting tubes (25 ml/flask) followed by seal and sterilization.

### Example 5

### The Growth State of OsSRO1c Transgenic Overexpression Rice Under Osmosis Stress

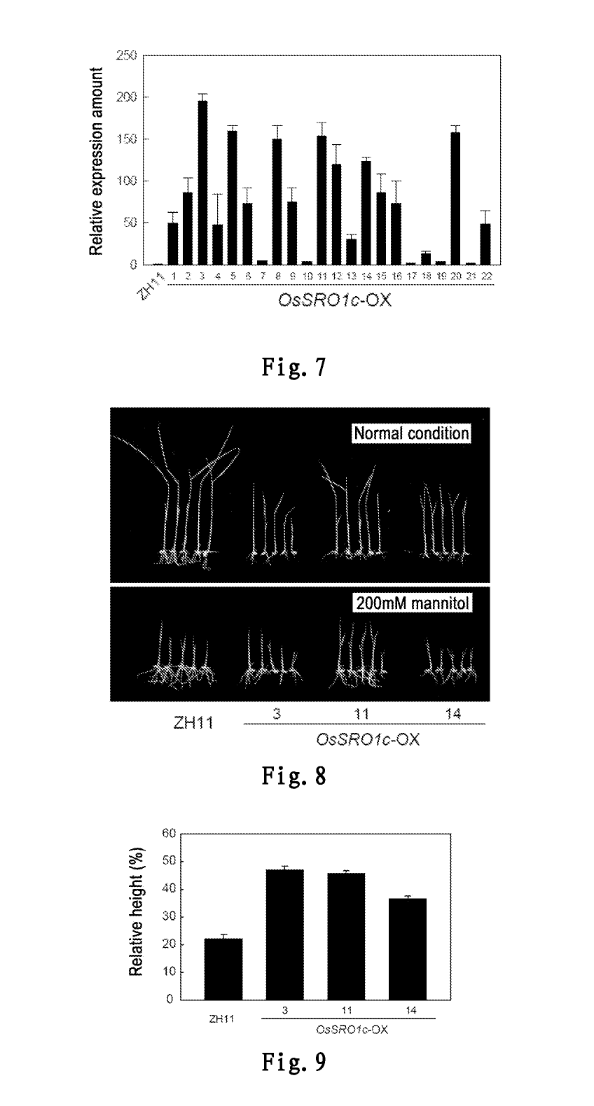

In the present invention, the expression of OsSRO1c gene in the transgenic rice plant obtained in the above step 4 was detected using fluorescent real time quantitative method. The particular steps regarding RNA extraction, reverse transcription and fluorescent real time PCR are the same as those in example 1. The expression results are shown in FIG. 7, demonstrating that the expression amounts of OsSRO1c gene in most transgenic plants are enhanced compared with those of the wild type lines.

Three OsSRO1c (whose sequence is set forth in SEQ NO: 1) transgenic and overexpression T1 lines (OsSRO1c-OX-1,-11,-14) were selected and treated with mannitol, simulating the osmosis stress caused by drought to determine the drought resistance of the transgenic rice. The experiments were carried out as follows: the seeds of the transgenic and overexpression lines (OsSRO1c-OX-1,-11,-14) were deshelled and then sterilized (treated with 70% alcohol for 1 minute; disinfected with 0.15% HgCl₂ for 10 minutes and washed with sterilized water for several times). The seeds were germinated in ½ medium in the presence of 50 mg/L hygromycin and Zhonghua 11 (ZH11) lines were seeded in ½ MS medium absent of hygromycin one day later.

2-3 days later, well germinating seeds with consistent germinating rate were picked and transferred into ½ MS medium with or without 200 mM mannitol for further development. 10 days later, the heights of the plants were observed (FIG. 8). Since the overexpression plants grew at a rate different from the control plants in the medium without mannitol, the relative plant height (i.e. the plant height in the medium with mannitol is divided by the plant height in the normal medium) is used to evaluate the resistance to osmosis stress of the plants. The relative height of the overexpression plants is approximately 2 times of that of the wild type plants (FIG. 9). For each line, the experiments were in biological triplet for each line and the results are consistent, showing the overexpression of OsSRO1c gene indeed enhances the drought resistance of the transgenic plants.

1-16. (canceled)

17. A DNA construct comprising a heterologous promoter operably linked to a polynucleotide encoding a SRO (SIMILAR-TO-RCD-ONE) protein comprising the amino acid sequence as set forth in SEQ ID NO:2.

18. The DNA construct of claim 17, wherein said polynucleotide comprises the nucleotide sequence as set forth in SEQ ID NO:1 from nucleotide position 77 to nucleotide position 1465.

19. The DNA construct of claim 18, wherein said polynucleotide comprises the nucleotide sequence as set forth in SEQ ID NO:1.

20. The DNA construct of claim 17, wherein said promoter is a drought inducible promoter.

21. A transgenic plant or plant part comprising the DNA construct of claim 17, wherein said plant is drought tolerant and said plant part comprises seed or cell.

22. The transgenic plant or plant part of claim 21, wherein said plant is selected from the group consisting of a corn, cotton, soybean, rice and wheat plant.

23. A method of improving drought resistance of a plant, comprising providing a transgenic plant comprising the DNA construct of claim 17, wherein expression of said polynucleotide is induced under drought condition thereby improving the drought resistance of the transgenic plant.

24. The method of claim 23, wherein said providing step further includes transforming a plant with said DNA construct thereby obtaining said transgenic plant.

25. The method of claim 23, wherein said plant is selected from the group consisting of a corn, cotton, soybean, rice and wheat plant.

26. A DNA construct comprising a heterologous promoter operably linked to a polynucleotide encoding a SRO protein comprising the amino acid sequence as set forth in SEQ ID NO:2, wherein said promoter is an ubiquitin promoter.

27. The DNA construct of claim 26, wherein said promoter is a corn ubiquitin promoter.

28. The DNA construct of claim 26, wherein said polynucleotide comprises the nucleotide sequence as set forth in SEQ ID NO:1 from nucleotide position 77 to nucleotide position 1465.

29. The DNA construct of claim 28, wherein said polynucleotide comprises the nucleotide sequence as set forth in SEQ ID NO:1.

30. A transgenic plant or plant part comprising the DNA construct of claim 26, wherein said plant is drought tolerant and said plant part comprises seed or cell.

31. The transgenic plant or plant part of claim 30, wherein said plant is selected from the group consisting of a corn, cotton, soybean, rice and wheat plant.

32. A method of improving drought resistance of a plant, comprising providing a transgenic plant comprising the DNA construct of claim 26, wherein said polynucleotide is overexpressed compared to a wild type plant, said transgenic plant exhibiting improved drought resistance compared to the wild type plant.

33. The method of claim 32, wherein said providing step further includes transforming a plant with said DNA construct thereby obtaining said transgenic plant.

34. The method of claim 32, wherein said plant is selected from the group consisting of a corn, cotton, soybean, rice and wheat plant.

35. A method of sowing, planting, or growing a plant with improved drought resistance, said method comprising the step of sowing, planting, or growing a transgenic plant comprising the DNA construct of claim 17.

36. A method of sowing, planting, or growing a plant with improved drought resistance, said method comprising the step of sowing, planting, or growing a transgenic plant comprising the DNA construct of claim 26.
